(19) United States
(12) Patent Application Publication
(10) Pub. No.: US 2006/0269601 A1
(43) Pub. Date: Nov. 30, 2006

(54) Oral modified release formulations containing 8-prenylnaringenin for continuous estrogen support

(76) Inventors: Michael Huempel, Berlin (DE); Heiko Kranz, Berlin (DE)

Correspondence Address: MAYER & WILLIAMS PC, 251 NORTH AVENUE WEST, 2ND FLOOR, WESTFIELD, NJ, 07090, US

(21) Appl. No.: 11/366,947

(22) Filed: Mar. 2, 2006

(30) Foreign Application Priority Data
Mar. 2, 2005 (EP) EP 05090049.7
Aug. 12, 2005 (EP) EP 05076899.3

(51) Int. Cl. A61K 31/353; A61K 9/22

(52) U.S. Cl. 424/468; 514/456

(57) ABSTRACT

This invention is directed to an oral modified release formulation of the phytoestrogen 8-prenylnaringenin and methods for the treatment of symptoms of estrogen deficiency.

### FIELD OF THE INVENTION

This invention is directed to oral modified release formulations containing 8-prenylnaringenin (8-PN) and its use in continuous estrogen support to treat estrogen deficiency conditions.

### BACKGROUND OF THE INVENTION

Estrogen deficiency conditions in females can have different causes, i.e. inactive or surgically removed ovaries or the cease of estrogen production during menopause. One medical intervention to treat such estrogen deficiency conditions is to replace the missing estrogens (estradiol, estrone, estriol) or to use other estrogens like ethinyl estradiol or conjugated (equine) estrogens. The main use of these compounds is to replace estrogen activity during menopause (Hormone Replacement Therapy, HRT). All these compounds are effective in the treatment of menopausal symptoms (short term) and the treatment and prevention of osteoporosis (short and long term). Recently, a new estrogen, 8-prenylnaringenin, found in plants (hop, Anaxagorea luzonensis A. Gray) was shown to be not only the most active phyto-estrogen but also a substance that exhibits tissue specificity with a much lower activity on the uterus than estradiol at equivalent bone protective doses. On the basis of this new pharmacological profile, it was claimed that menopausal symptoms and the treatment and protection of osteoporosis is possible without the concomitant administration of a progestin and thus without the induction of withdrawal bleeding (WO 2005/037816). 8-prenylnaringenin (5,7-Dihydroxy-2-(4-hydroxyphenyl)-8-(3-methylbut-2-enyl)-chroman-4-on) has the following structure:

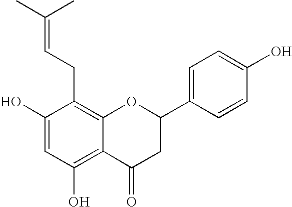

The 4-hydroxyphenyl group may either be in the 2S(−) or the 2R(+) position.

The normal route of administration in hormone replacement therapy is the oral route. This route is not only preferred because of user convenience but also because most of the substances in question require a high daily dose which is difficult to administer by alternate routes such as nasal, dermal or inhalatory routes. For the most potent natural estrogen, estradiol, the dermal route was shown to be an effective alternative to the oral route because only small quantities (25-100 μg/d) need to reach systemic circulation. There are two reasons why the effective daily oral dose (1 or 2 mg) can be reduced by a factor of 10-40 when estradiol is absorbed via the skin. Most importantly, the liver, which inactivates roughly 90% of the oral dose before reaching the systemic circulation, is bypassed by the dermal route. Thus, avoidance of the high first-liver-pass metabolism by dermal administration leads to a drastic reduction in the effective dose. In addition, the continuous and steady influx of drug (zero order kinetics) throughout the time a patch is adminstered (2-7 days) has a dose sparing effect as compared to the oral route, which is typically characterized by a mixture of several first order kinetics, i.e. absorption, distribution and disposition processes starting at a predetermined time of day. This leads to steep drug serum level increases shortly after intake and subsequent decreases governed by distribution and metabolism/excretion.

Because all marketed estrogens, natural or synthetic, undergo an almost complete metabolization before excretion and show high intrinsic clearance rates, drug serum levels decrease rapidly and generally drop to very low and sometimes undetectable levels within the treatment interval of 1 day. As a result, serum (and subsequently tissue) concentrations show high fluctuations with periods of maximum effective levels and below threshold effective levels. At the doses used for marketed estrogens, it can be assumed that serum and tissue concentrations fell below a maximum effective level for several hours a day. It is assumed that an effective dose can be lowered by 30-50% when the total dose is fairly distributed over the total treatment interval.

A potential solution to this problem could be the use of an oral modified release formulation. These pharmaceutical formulations were developed for other drugs to avoid high plasma fluctuations of the active compound.

The question, however, arises as to why no modified release formulations are available on the market for hormone replacement therapy. The answer is that modified release formulations need higher total doses for substances undergoing a high first-liver-pass metabolism because there is a risk that the liver would inactivate even higher percentages of the total dose when they reach the liver at small doses for an extended period of time. In addition, a modified release formulation can only deliver an active ingredient for the duration of gastro-intestinal transit time, which is highly variable but takes 12-16 hours on average. Thus, a modified release formulation containing one of the marketed estrogens would not be able to continuously replace estrogen 24 hours a day.

The problem, therefore, is to provide an oral formulation for 8-prenylnaringenin which continuously distributes 8-prenylnaringenin for almost 24 hours a day and which preferably has to be administered only once a day.

### SUMMARY OF THE INVENTION

This problem was solved by providing a solid oral modified release formulation of 8-prenylnaringenin for oral delivery. This formulation comprises a pharmaceutical composition containing a polymeric matrix, a buffer substance and one or more excipients in addition to 8-prenylnaringenin. Preferably the buffer substance is an alkaline substance such as, for example, magnesium oxide, magnesium hydroxide, dihydroxyaluminum aminoacetate, magnesium carbonate, calcium carbonate, sodium ascorbate, magnesium trisilicate, dihydroxyaluminum sodium carbonate, aluminum hydroxide, sodium citrate, potassium phosphate, sodium bicarbonate, disodium hydrogenphosphate or some combinations thereof. Preferably, the particle size of the compounds is in the range of about 0.1-750 μm. Most preferably it is in the range of about 20-400 μm. The polymer matrix is preferably selected from the group consisting of: cellulose derivatives, acrylic derivatives, vinyl polymers, polyacrylates, polycarbonates, polyethers, polystyrenes polyanhydrides, polyesters, polyorthoesters, polysaccharides and natural polymers. Most preferably, the polymer matrix comprises water soluble polyvinylpyrrolidone and/or water insoluble polyvinylacetate. In a preferred embodiment, the oral modified release formulation is coated with a polymeric coating.

Excipients can comprise lactose, calcium phosphate, manitol and starch. Preferably, an additional excipient is microcrystalline cellulose. In addition, the formulations can further comprise a flow promoter such as silicon dioxide and any pharmaceutically acceptable carriers.

The solid oral modified release formulations of this invention show a pH-independent drug release in vitro. This is important as the pH varies considerably in the gastro-intestinal tract and continuous release should be achieved independent of the pH. However, the solubility of 8-prenylnaringenin is pH-dependent. The compound shows higher solubility at higher pH-values whereas the solubility of the compound is low at lower pH-values. The low acid solubility property of 8-prenylnaringenin results in vitro in slow drug dissolution at pH 1, whereas the dissolution is fast at higher pH-values such as pH 6.8. The resulting dissolution profiles are different at different pH-values. This problem is solved by the solid oral modified release formulation of this invention.

The solid oral modified release formulation of this invention solves the problem of a continuous distribution of 8-prenylnaringenin almost over 24 hours. This is a combined effect of the pharmacokinetic profile of 8-prenylnaringenin, which is drastically different from those of other estrogens, and the solid oral modified release formulation.

The pharmacokinetic profile of 8-prenylnaringenin is characterized by a complete oral absorption, a low degree of metabolization (less than 60% of dose) and a high pre-systemic elimination (first-liver-pass excretion; about 55% of dose). This high and unexpected pre-systemic elimination leads to an accumulation of substantial amounts in the bile fluid from which they are secreted into the duodenum when gastric signaling occurs with food intake. The 8-prenylnaringenin is then absorbed again and enters systemic circulation. Examples for this effect (drug serum levels) are shown in FIG. 1. Data were taken from the clinical Phase Ia study and represent two volunteers in the lowest dose group (50 mg 8-prenylnaringenin).

Taking the total area under the curve as a measure of 8-prenylnaringenin systemic availability, the percentage of the area under the second peak (generated by reabsorption of the pre-systemically eliminated and then biliary secreted amounts) can be used to estimate those portions of the doses that undergo pre-systemic elimination after oral dosing. The average figure was 54±20% for the six women in the study.

The invention is to combine this unexpected pharmacokinetic property of 8-prenylnaringenin with a modified release formulation, which can release the drug at an almost constant rate for 8-10 hours. The drug release from the solid oral modified release formulation according to this invention preferably follows zero-order kinetics or near zero-order kinetics. This oral modified release formulation is preferably taken in the evening (after the evening meal or prior to bedtime). Nighttime release of 8-prenylnaringenin occurs at an almost constant rate leading to flat drug serum levels and an accumulation of substantial amounts in the bile fluid which—after reabsorption—account for more than half of the total systemically available dose. On the following day, the accumulated drug is secreted into the duodenum when the first meal (breakfast or lunch) is taken. Reabsorption gives rise to elevated 8-prenylnaringenin serum levels during the first half of the day, which at a lower level, occurs again following intake of the evening meal. Overall, the total systemically available dose is distributed over 24 hours thereby avoiding unnecessary high peaks and ensuring effective drug concentrations in target tissues. The anticipated daily dose is between about 50 and 250 mg, preferably between about 75 and 150 mg. The oral modified release formulation is preferably taken 6-12 hours, more preferably 8-12 hours before a meal.

In yet another aspect, this invention provides a method of treating a human patient who is suffering from estrogen deficiency conditions by administering a solid oral modified release 8-prenylnaringenin formulation of the invention to the patient twice or preferably once daily. The invention is also related to the use of said oral modified release formulations for the production of a medicament for the treatment of symptoms of estrogen deficiency or for the treatment of menopausal symptoms. Said symptoms can be hot flashes, night sweat, mood disturbances or osteoporosis.

In still another aspect, this invention provides a method of maintaining therapeutically effective levels of 8-prenylnaringenin in human plasma by administering an 8-prenylnaringenin containing oral modified release formulation twice or preferably once daily.

The method includes administering an oral modified release formulation including from about 5 to about 80% by weight 8-prenylnaringenin in no more than two dosage forms per dose to the human patient, to maintain 8-prenylnaringenin plasma levels in the human patient from about 0.5 to about 5 ng 8-prenylnaringenin/ml for at least 24 hours wherein the dose is administered at a frequency selected from twice or preferably once a day.

The formulations of this invention may be either in the form of single unit dosages, e.g. tablets, or in the form of multiple unit dosages, e.g. granulates, pellets or mini-tablets. These multiple unit dosages may be filled in gelatin capsules or compressed into tablets.

The single unit dosages may be produced by powder blending and direct compression into tablets or by powder blending, granulation and compression into tablets. The tablets may be coated by a film.

Multiple unit dosage may be produced by extrusion/spheronization, by layering technique, by rotor granulation, by powder blending and direct compression into mini tablets, by powder blending, granulation and compression into mini tablets, by powder blending, direct compression into mini tablets and coating with a film or by powder blending, granulation, compression into mini tablets and coating with a film.

8-prenylnaringenin can be produced by the method described in WO 2005/037816.

### BRIEF DESCRIPTION OF THE FIGURES

FIG. 1 shows drug serum levels following a single oral dose of 50 mg 8-prenylnaringenin in two postmenopausal women; 8-prenylnaringenin was administered as a 1:1 mixture with lactose (gelatin capsule) at fasted state in the morning, with intake of a midday meal six hours later. Re-increases in drug serum levels show reabsorption of dose parts collected in the bile fluid and subsequently secreted triggered by intake of the midday meal.

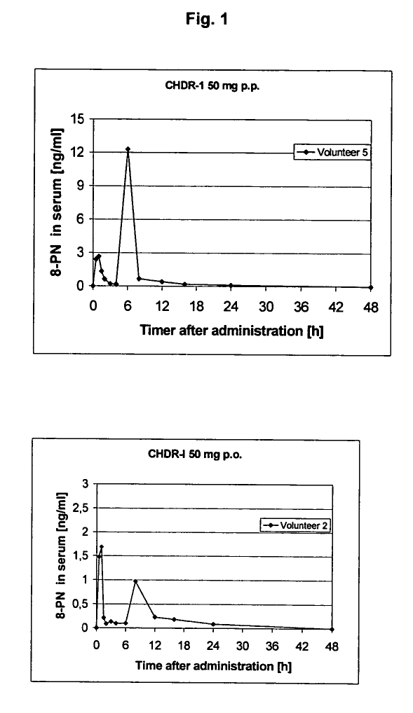

FIG. 2 shows the pH-dependent solubility of 8-prenylnaringenin.

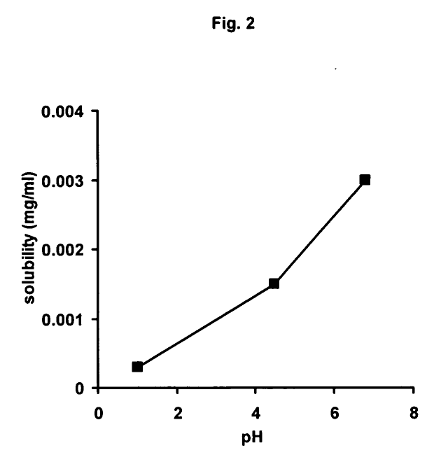

FIG. 3 shows the pH-dependent release of 8-prenylnaringenin from mini matrix tablets prepared without a buffer substance (according to Example 1).

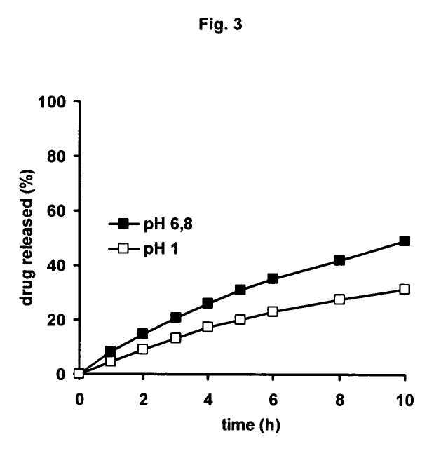

FIG. 4 shows the pH-independent release of 8-prenylnaringenin from mini matrix tablets prepared after the addition of magnesium oxide (according to Example 2).

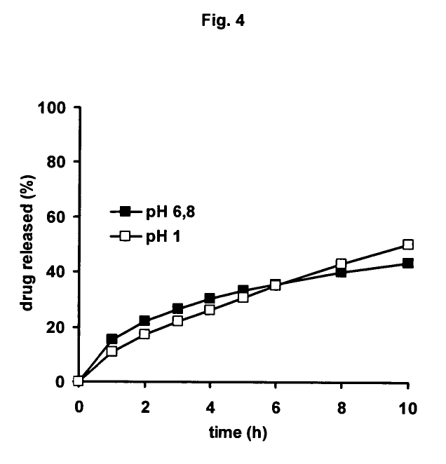

FIG. 5 shows the pH-independent release of 8-prenylnaringenin from mini matrix tablets prepared after the addition of magnesium hydroxide (according to Example 3).

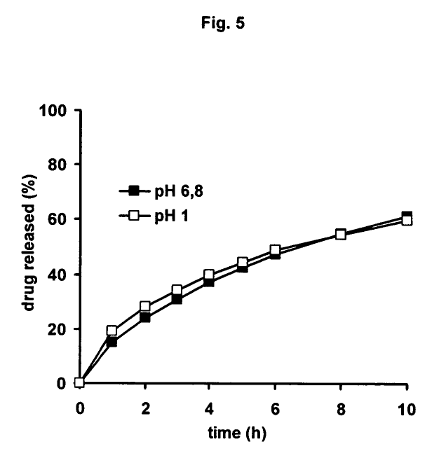

FIG. 6 shows the renal excretion rate of 8-prenylnaringenin conjugates (% of total recovery per hour) as determined for various urine collection periods (mid-points of periods taken as determinants) following single oral administration of 25 mg of 8-prenylnaringenin as an alcoholic solution or as a modified release formulation. Each volunteer ingested formulations at night (9:30 p.m.) and treatments were one week apart. Renal excretion of drug conjugates has been shown to be a reliable surrogate parameter for systemic drug availability in the clinical Phase I study.

### DETAILED DESCRIPTION OF THE PREFERRED EMBODIMENTS

### EXAMPLES

### Example 1

Preparation of a Modified Release Formulation of 8-prenylnaringenin

Production of Mini-Matrix Tablets by Means of Direct Tableting,
2.333 mg of 8-prenylnaringenin
1.000 mg of KOLLIDON SR® (registered trademark of BASF Akfiengesellschaft, Germany, for polyvinylpyrrolidone)
1.992 mg of lactose
1.500 mg microcrystalline cellulose
0.070 mg of highly dispersed silicon dioxide
0.105 mg of magnesium stearate

8-prenylnaringenin, KOLLIDON SR®, lactose and microcrystalline cellulose are sieved individually and mixed in a TURBULA® mixer (registered trademark of Glenn Mills, Inc., Clifton, N.J., for an oscillo-rotary mixer-shaker) for 10 minutes. Highly dispersed silicon dioxide, sieved, is added, and all components are mixed in the mixer for another 5 minutes. Magnesium stearate, sieved, is spread on, and all components are mixed in the mixer for another 30 seconds. Tableting of the powder mixture into mini-matrix tablets is carried out by means of an eccentric tablet press or a rotary tablet press.

The release from these mini-tablets is measured by means of the method that is mentioned in Example 4.

### Example 2

Production of Mini-Matrix Tablets by Means of Direct Tableting
2.333 mg of 8-prenylnaringenin
1.000 mg of KOLLIDON SR®
1.169 mg of magnesium oxide
0.823 mg of lactose
1.500 mg microcrystalline cellulose
0.070 mg of highly dispersed silicon dioxide
0.105 mg of magnesium stearate

8-prenylnaringenin, KOLLIDON SR®, magnesium oxide, lactose and microcrystalline cellulose are sieved individually and mixed in a TURBULA® mixer for 10 minutes. Highly dispersed silicon dioxide, sieved, is added, and all components are mixed in the TURBULA® mixer for another 5 minutes. Magnesium stearate, sieved, is spread on, and all components are mixed in the mixer for another 30 seconds. Tableting of the powder mixture into mini-matrix tablets is carried out by means of an eccentric tablet press or a rotary tablet press.

The release from these mini-tablets is measured by means of the method that is mentioned in Example 4.

### Example 3

Production of Mini-Matrix Tablets by Means of Direct Tableting
2.333 mg of 8-prenylnaringenin
1.000 mg of KOLLIDON SR®
1.169 mg of magnesium hydroxide
0.823 mg of lactose
1.500 mg microcrystalline cellulose
0.070 mg of highly dispersed silicon dioxide
0.105 mg of magnesium stearate

8-prenylnaringenin, KOLLIDON SR®, magnesium hydroxide, lactose and microcrystalline cellulose are sieved individually and mixed in a TURBULA® mixer for 10 minutes. Highly dispersed silicon dioxide, sieved, is added, and all components are mixed in the mixer for another 5 minutes. Magnesium stearate, sieved, is spread on, and all components are mixed in the TURBULA® for another 30 seconds. Tableting of the powder mixture into mini-matrix tablets is carried out by means of an eccentric tablet press or a rotary tablet press.

The release from these mini-tablets is measured by means of the method that is mentioned in Example 4.

### Example 4

Measurement of the Release of 8-prenylnaringenin

Measurement of the active ingredient release from mini-matrix tablets is carried out according to a one-compartment method (basket apparatus), as described in U.S. Pharmacopeia USP XXV. The release of 8-prenylnaringenin was examined in phosphate buffer solution, pH 6.8 (composition, see USP XXV) or in 0.1 N HCl. Ten percent (w/w) hydroxypropyl-β-cyclodextrine were added in order to achieve sink conditions and primarily control the drug release by the dosage form.

### Example 5

Method for Quantitative Analysis of 8-prenylnaringenin in Biological Matrices

8-prenylnaringenin is analyzed by a specifically developed radio-immunoassay. A 4′-O hapten ({4-[5,7-Dihydroxy-8-(3-methyl-but-2-enyl)-4-oxo-chroman-2-yl]-phenoxy}-acetic acid) was synthesized starting from racemic naringenin and coupled to cationized bovine serum albumin (cBSA). This antigen was mixed with Freund's adjuvants and injected into rabbits. The antiserum resulting after several boosters was isolated as an IgG fraction and used in a final dilution of 1:100.000. A tritiated tracer was synthesized by preparation of the 3′,5′-dibromo derivative of 8-prenylnaringenin, again starting from racemic naringenin. Both bromine atoms were exchanged to ³H in a final, palladium-catalyzed step leading to the tritiated tracer of a specific radioactivity of 2.22 GBq/mg.

Biological matrices, i.e. blood plasma, blood serum, or urine, are either directly extracted with tert-butyl methyl ether (tert-BME) or after enzymatic de-conjugation by means of glucuronidase/arylsulfatase (Helix pomatia). Organic layers are separated, evaporated and the residues taken up in assay buffer. Extract residues or dilutions of standard 8-prenylnaringenin solutions are then mixed with antiserum and tracer and kept at 4° C. overnight. Dextran-coated charcoal is added to separate bound and free 8-prenylnaringenin and bound radioactivity is quantified with a liquid scintillation counter after addition of a scintillation cocktail.

### Example 6

Comparison of an Acute Liberating and a Modified Releasing Formulation (MRF) in an In-Vivo Human Renal Excretion Study.

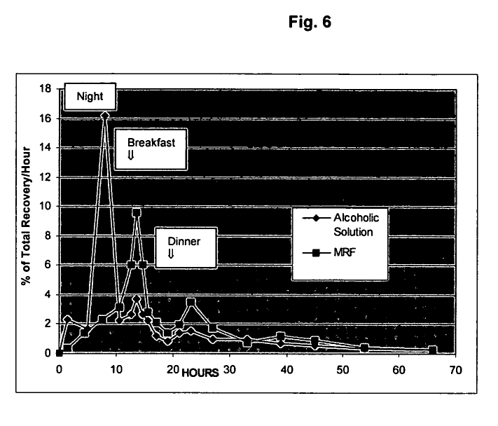

An aqueous/alcoholic solution (44% (v/v) ethanol, 2.5 mg 8-Prenylnarin-genin/ml) and a formulation as described in Example 3 (7 mini tablets with 50 ml tap water) were taken at a time for a period of one week. Both preparations contained 25 mg 8-prenylnaringenin and were taken in the evening 9:30 p.m., 12 hours prior to the intake of a full breakfast. After breakfast, a fasting period of 7-8 hours followed before an evening meal was served. The second day after treatment followed a similar dietary routine. Urine samples were collected at different time intervals before and after the administration of formulations, their volumes were recorded and aliquots kept at −16° C. until analysis. Urine samples covered the complete excretion over three days after treatment (2×20 samples). Each 0.5 ml of urine were hydrolyzed with glucuronidase/arylsulfatase, aliquots extracted with tert-BME, extracts evaporated (N₂), and extract residues dissolved in an assay buffer. Each sample was extracted in duplicate and measured by the method described in Example 5. Results were converted into percentage of total 8-prenylnaringenin recovery per collection period and into % of total recovery per hour within the collection periods in order to get directly comparable figures. Excretion rates are displayed in time dependency using the mid point of collection periods as the determinant of the y-axis. FIG. 6 provides the results and clearly shows that renal excretion mirrors the systemic availability of 8-prenylnaringenin. Following treatment with the aqueous/alcoholic solution, the excretion rate peaked before breakfast while the MRF released the drug slowly overnight leading to slowly increasing excretion rates. Reabsorption processes after breakfast and the evening meal became visible after both treatments but the modified release formulation markedly shifted the area under the curve to daytime. Thus, the aim to fairly distribute 8-prenylnaringenin availability over one full treatment interval of 24 hours was reached by the combination of a modified release formulation taken at nighttime and the enterohepatic recirculation of 8-prenylnaringenin occurring at daytime after food uptake (breakfast, midday meal, evening meal).

The term “modified release” is defined in the European Pharmacopoeia as a modification of the rate or the place at which the active substance is released. Modified release products cover a wide range of release models, the principle types of which include “delayed release” and “prolonged release” products. In this specification, the term modified release relates to pharmaceutical dosage forms that show prolonged (=extended) release of an active substance from solid oral dosage forms.

Accordingly, a prolonged release product is a product, in which the rate of release of active substance from the formulation after administration has been reduced, in order to maintain therapeutic activity, to reduce toxic effects and/or for some other therapeutic purpose.

In contrast, a conventional release dosage form is a preparation, wherein the release of the active ingredient is not modified by a special formulation and/or manufacturing method. In case of a solid dosage form, the dissolution profile of the active ingredient depends essentially on the intrinsic properties of the active ingredient. Conventional release dosage forms are also called immediate release dosage forms.

In summary, a prolonged release product shows a reduced release rate, compared to a product with the same active ingredient but without formulation components being effective to reduce the release rate.

1. A modified release pharmaceutical composition for oral delivery comprising 8-prenylnaringenin, a polymeric matrix, a buffer substance and one or more excipients in a solid form.

2. The pharmaceutical composition of claim 1, wherein the solid form is prepared from a powder mixture of 8-prenylnaringenin, a polymeric matrix, a buffer substance and one or more excipients, wherein the powder mixture comprises particles having a size in the range of about 0.1 to about 750 μm.

3. The pharmaceutical composition according to claim 2, wherein the particle size of the powder mixture is between about 20-400 μm.

4. The pharmaceutical composition according to claim 1, wherein the buffer substance is an alkaline substance.

5. The pharmaceutical composition according to claim 1, wherein the solid form is coated with a polymeric coating that affects the dissolution of 8-prenylnaringenin.

6. The pharmaceutical composition according to claim 1, wherein the polymer matrix is selected from the group consisting of: cellulose derivatives, acrylic derivatives, vinyl polymers, polyacrylates, polycarbonates, polyethers, polystyrenes polyanhydrides, polyesters, polyorthoesters, polysaccharides and natural polymers.

7. The pharmaceutical composition according to claim 1, wherein the polymer matrix comprises water soluble polyvinylpyrrolidone or water insoluble polyvinylacetate.

8. The pharmaceutical composition according to claim 1, wherein the buffer substance comprises magnesium oxide, magnesium hydroxide, dihydroxyaluminum aminoacetate, magnesium carbonate, calcium carbonate, sodium ascorbate, magnesium trisilicate, dihydroxyaluminum sodium carbonate, aluminum hydroxide, sodium citrate, potassium phosphate, sodium bicarbonate, disodium hydrogenphosphate or combinations thereof.

9. The pharmaceutical composition according to claim 1, further comprising a lubricant.

10. The pharmaceutical composition according to claim 1, wherein the excipient comprises lactose, calcium phosphate, mannitol or starch.

11. The pharmaceutical composition according to claim 1, wherein one of the excipients comprises microcrystalline cellulose.

12. The pharmaceutical composition according to claim 1, further comprising a flow promoter comprising silicon dioxide.

13. The pharmaceutical composition according to claim 1, wherein the polymeric matrix comprises polyvinylpyrrolidone, the buffer substance comprises magnesium oxide or magnesium hydroxide, and the excipient comprises lactose.

14. The pharmaceutical composition according to claim 13, further comprising microcrystalline cellulose as an excipient.

15. The pharmaceutical composition according to claim 1, wherein the solid form comprises granulates, pellets, tablets, or mini-tablets.

16. The pharmaceutical composition according to claim 1, wherein the solid form comprises tablets having a coating comprising a polymer film.

17. The pharmaceutical composition according to claim 1, wherein the solid form comprises granulates, pellets, or mini-tablets contained in gelatin capsules.

18. A method of treating symptoms of estrogen deficiency in a human patient comprising administering the modified release pharmaceutical composition of claim 1 by oral delivery once or twice daily.

19. The method of claim 18, wherein the estrogen deficiency is caused by a menopausal condition and symptoms comprise hot flashes, night sweat, mood disturbances or osteoporosis.

20. The method of claim 18, wherein the pharmaceutical composition further comprises a coating on the solid form that affects the dissolution of 8-prenylnaringenin.
